(19) United States
(12) Patent Application Publication
(10) Pub. No.: US 2008/0287811 A1
(43) Pub. Date: Nov. 20, 2008

(54) Method for Assessing The Functional Condition Of Cardiovascular System

(76) Inventors: Vladimir Petrovich Nesterov, Saint Petersburg (RU); Anton Igorevich Burdygin, Saint Petersburg (RU); Sergei Vladimirovich Nesterov, Saint Petersburg (RU)

Correspondence Address: WALTER J. TENCZA JR., 100 Menlo Park, Suite 210, Edison, NJ, 08837, US

(21) Appl. No.: 10/599,919

(22) PCT Filed: Apr. 14, 2005
(86) PCT No.: PCT/RU05/00198

§ 371 Date: May 2, 2007

(30) Foreign Application Priority Data
Apr. 14, 2004 (RU) 2004112563

(51) Int. Cl. A61B 5/02

(52) U.S. Cl. 600/485

(57) ABSTRACT

The invention relates to medicine, namely: to cardiology, and may be used for assessment of functional condition of the human cardiovascular system (CVS) and the character of its control by the autonomic nervous system and other regulatory systems of the homeostasis. A method of non-invasive examination of the human CVS was developed, the method enabling to continuously, during a necessary period of time and quite simply with the aid of a computer and a piezoceramic tranducer (FIG. 3), record differential sphygmograms (FIG. 4) and by these sphygmograms using the method of determining the “coding” points to perform express-analysis simultaneously of two main pulse characteristics: a) rhythmicity and b) pulse oscillation of the arterial pressure (AP). The automatic disposition of the “coding” points in the averaged graph of the cardiocycle and their additional visual correction (FIG. 5) guarantee precision of determining the amplitude-temporal parameters at each recognised normal pulsation of a selected pulsogram fragment. By this fragment, the cardiac rhythm and all the amplitude-temporal cardiohemodynamic parameters will be measured and analysed, the parameters characterising the left ventricle myocardium contractile capacity as well as the resilient-elastic properties of the arterial bed vessel walls. For this purpose, the conventional units of the computer “digitizing” will be calibrated and transformed into accepted units of the blood AP measurement (mm Hg) and then, by means of integrating by respective areas of the cardiocycle graphs, the values of the blood AP pulse increment will be determined for different stages of the cardiac cycle. The continuous monitoring of the pulsogram parameter changes provides fulfillment of spectral analysis of the cardiac rhythm variability as well as of the selected cardiohemodynamic parameters. By results of the statistical and spectral analyses of the measured parameters' variability, the functional condition and the character of the subject's CVS vegetative regulation will be assessed by comparing the obtained values with the average statistical numerical values of these same parameters established for the CVS of groups of people who were selected as control subjects. The results may be used for resolving the problems of differential diagnosis of the cardiovascular diseases under clinical conditions, for individual examination of patients as well as for performing an operative medical checkup of health condition in various groups of population.

### FIELD OF THE INVENTION

The invention relates to medicine, namely, to cardiology, and may be used for non-invasive express-analysis of the functional condition of the human cardiovascular system (CVS) and the character of its control by the autonomic nervous system (ANS) and other regulatory systems of the homeostasis. On the basis of the invention, a new diagnostic device was developed for complex and at the same time simple examination of the human CVS using computer recording and analysis of the heart rhythm and oscillations of arterial walls during pulse wave passage. The invention may be used for diagnosis of cardiovascular diseases in clinics, in operative medical checkups of the health condition in various groups of population, and in medical prognostic studies for assessment of trends in development of functional pre-clinical changes in the CVS and the probability of their spreading beyond admissible limits.

### BACKGROUND OF THE INVENTION

The development and improvement of methodology and technical means for early diagnosis of the human CVS condition is an extremely urgent task at present, as this particular system is the most vulnerable part of the organism when subjected to physical and emotional (stress) loads, and just these cardiovascular pathological changes occupy a stable first place in the morbidity, physical inability and mortality structure in developed countries. The autonomic nervous system plays a most important role in the control of the CVS and in adaptation of its functions to changing conditions of environment and inner milieu. That is why modern systems of complex examination of the CVS should also include the assessment of the character of vegetative regulation of this system.

Until recently, the systems of such examination of the CVS have been mainly based on the variation-statistical and spectral analysis of the cardiointervalogram obtained with the aid of the electrocardiography technique (ECG) (e.g., such well known systems as “Ankar”, “Inkart”, “Holter for Windows”, “SphygmoCor Px”, etc.).

There is a patented technique for diagnosis by the cardio-rhythm using the ECG for recording and accumulation of cardiointervals for a certain time epoch with their subsequent analysis [1]. However, the ECG technique used in such systems and means, in spite of its high informative value for studying the heart electrical excitation pattern and its wide usability in spectral analysis of the heart rhythm variability, is unable to sufficiently assess the cardiac dynamics, the myocardium contractile capacities or the condition of vascular tone. At the same time, in patients, functional disorders occurring in the myocardium and blood vessels often precede the changes revealed with the aid of the ECG. Therefore, in recent years, a few systems have been actively developed which also use other ways of non-invasive study of the CVS condition.

The technique of ultrasonic echocardiography has widely spread as it enables one to perform a non-invasive assessment of a number of important cardio- and hemodynamic characteristics of the CVS. Nonetheless, use of this technique requires complicated and expensive equipment, high quality of the operator, as well as a considerable duration of the examination process which reduces the technique's significance for obtaining express-information.

Further progress in this direction has been related to creation of specialized systems of the CVS analysis based on recording the amplitude-temporal parameters of the pulse waves in the form of electrical signals resulting from transformation of mechanical signals by special transducers, the signals originating from shifting of artery walls under the effect of the pulse pressure wave: the sphygmography (SPG), or from changing tissue volume under the effect of blood pulsing inflow: the plethysmography. On the basis of photo- and impedance plethysmographic and other transducers (e.g., using a compression cuff), such systems have been developed as “DynaPuls”, “Finapres”, “Portapres”, etc. [2], uniting many aspects of plethysmography and sphygmography, while the technique itself has been named a volumetric sphygmography (VSPG). At the same time, high cost of these devices and complexity of the procedure of deciphering the results still exist. For instance, when using a relatively inexpensive (499 USD) system “DynaPuls”, it is necessary to transfer the primary information via the Internet to a special commercial analytical centre in California for its interpretation, which generates additional difficulties and considerably increases the price of the examination.

Close to the proposed invention is the “Technique of assessment of the cardiovascular system functional condition” which is fulfilled by means of measuring the blood arterial pressure (AP) and recording the SPG during a single respiratory cycle in order to determine the average duration of a single cardiocycle and the time of pulse pressure increment (t, ms) [3]. This technique using the proposed empirical formulas enables one to approximately assess, by the value “t”, the diastolic and pulse pressure, in conventional units, the obviousness of functional stress under the effect of physical exercise.

However, it cannot be used in place of the CVS complex examination method because of a limited number of the parameters under study and impossibility of revealing the pattern of changes of the important for diagnosis parameters in the time course, being limited in analysis by just several pulse waves of a single respiratory cycle. Because of the same reason, the patented method cannot be used for the spectral analysis of variability of the amplitude-temporal parameters when determining the characteristics of the CVS vegetative regulation.

Essentially the closest to the method according to the proposed invention is the method using transducers for recording the VSPG with subsequent mathematical differentiation of the pulse curves [4]. This method's disadvantage involves the fact that the recorded signal reflects pulse changes of the arterial, capillary, and venous blood filling of the tissues, all three parameters changing their volume in different ways. This results in decrement of the signal, in evening-out or, on the contrary, in meshing of the contour of the cardiocycle graph, as well as in loss of a number of essential details in the recorded curve. Differentiation of such a pulsogram does facilitate the procedure of temporal analysis of the graph by the “coding” points but does not improve the precision or informative value of the examination, which further leads to uncertainty of assessment of the CVS condition, while the limited number of recorded cardiocycles does not allow for analysis of the organism regulatory systems' effect upon the CVS condition.

Analysis of the existing condition of the CVS pulse diagnosis problem has led to the preference for and the advantageous use of generator (induction and piezoelectric) transducers for direct recording of differential sphygmograms (DSPG) from the pulsing area of the body above the artery [5]. This has become possible in recent years due to industrial construction of compact and highly sensitive piezoelectric transducers with a wide band of operative frequencies and high own resonant frequency (over 2000 Hz) [6, see Addendum 3]. Such transducers are most precise and enable one to transform mechanical effects on the transducer directly into an analogue electrical signal that can be recorded in a graphic way in the form of a curve of speed of changing of the effect strength. Advancement of computer technology opened up a possibility of overcoming the obstacles occurring in quantitative processing and in analysis of large arrays of the pulsometric information obtained [7]. Continuous monitoring of the amplitude-temporal parameter changes in the pulsogram has become possible as well as obtaining of calculated data practically on-line and fast performance of complicated mathematical transformations for revealing the periodic components in oscillations of the amplitude-temporal parameters in the pulse curves in order to assess the significance of their contribution to provision of the necessary cardiohemodynamic patterns.

### DISCLOSURE OF THE INVENTION

The objective of the invention is creation of the method of non-invasive examination of functional condition of the human CVS enabling one to precisely, continuously, during a necessary period of time and in an uncomplicated way record the pulse curves and then to perform with them simultaneous express-analysis of two main characteristics of the heart rate: a) its rhythmicity and b) pulse oscillations of the arterial pressure induced by periodic output of the blood stroke volume into the aorta. For this purpose, a computerised version of the DSFG technique has been developed using a transducer in a form never used before in this field, a simple and convenient device for fastening it to the pulsating area of the body, the device being industrially manufactured (a sonic transformer of the Russian “3Π” type with a metal membrane having a piezoceramic element glued to the internal side, the element providing the transducer sensitivity about 0.5 mm Hg/s at its own resonance frequency of over 2600 Hz). A specially developed software (SW) and data processing algorithm made it simple for the operator (as well as for any person having even no medical qualification but strictly following the user's guide) to perform, in an automated mode, recording of the DSPG curve, measurement of the pulsogram amplitude-temporal parameters by the pulsogram selected fragment, and obtaining results of analysis of a wide range of parameters jointly characterising the CVS functional condition and specifics of its regulation by the VNS and other regulatory systems.

### BRIEF DESCRIPTION OF THE DRAWINGS

The invention will be better understood with reference to drawings and graphs in which:

FIG. 1—block diagram of the structural scheme of the device for pulsometric examination

FIG. 2—flow diagram of the functional scheme of the device for conjugating the computer signal

FIG. 3—block diagram for the data processing algorithm

FIG. 4—image of a recorded copy of the complete pulsogram and its selected fragments

FIG. 5—image of a recorded copy of the DSPG fragment with isolated set of individual pulsations and the graph of the averaged cardiocycle

FIG. 6—the three main types of the cardiocycle graphs

### PREFERRED EMBODIMENT OF THE INVENTION

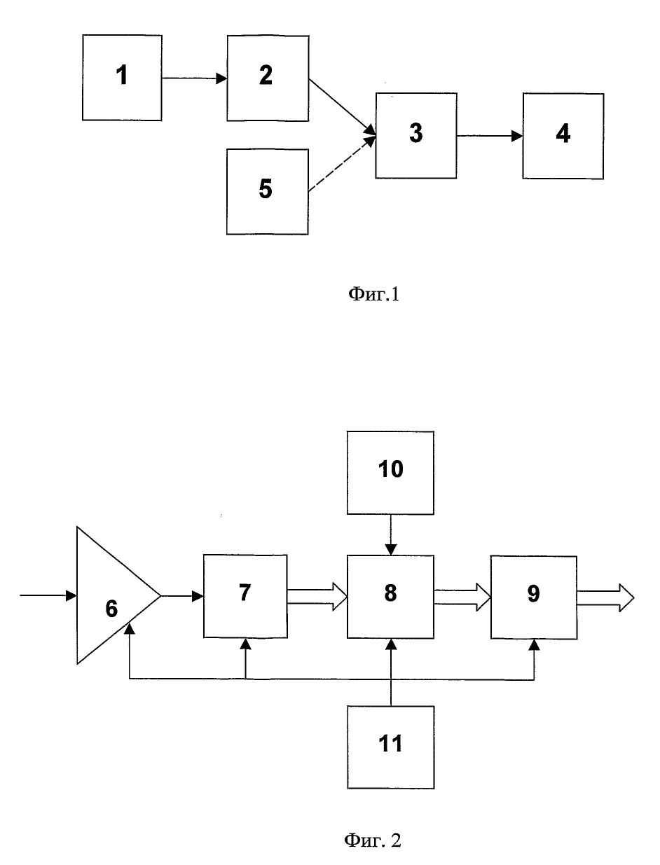

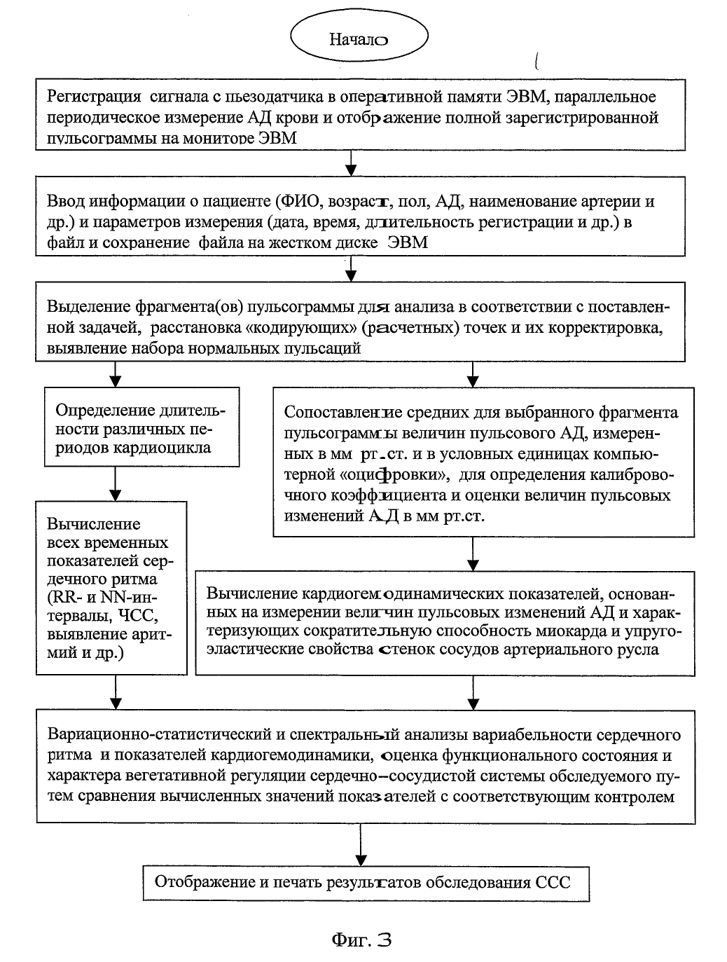

The device for implementing the method of the cardiovascular system pulsometric examination contains (FIG. 1) piezotransducer 1 whose output is connected to the input of the conjugating device 2 in its turn connected to the computer 3. The information will be displayed on the monitor 4. The second input of the computer 3 is connected to the output of the sphygmomanometer 5. The conjugating device 2 consists of an amplifier 6 (FIG. 2) whose output is connected with the input of the analog-digital transformer 7 (ADT) connected with the transformation block 8 whose output via the synchronizer unit 9 is connected to the computer. The step impulses from the output of the step impulses generator 10 (TPG) will be delivered to the transformation block 8. The power supply of the whole scheme will be provided by the power source 11. The analog signal from the transducer amplified to the necessary amplitude will go to the ADT input where it will be quantized with a certain discrete frequency (in our device, the frequency of 200 Hz is used and, respectively, the time interval between counts or the quantization increment duration−Δt=5 ms) and digitized. Further on, the information will be transferred to the transformation block 8 that by the step impulses from the TPG generates signals controlling the ADT and prepares data to be transferred via the synchronizer unit 9 by the serial link to the computer's estimator with respective SW.

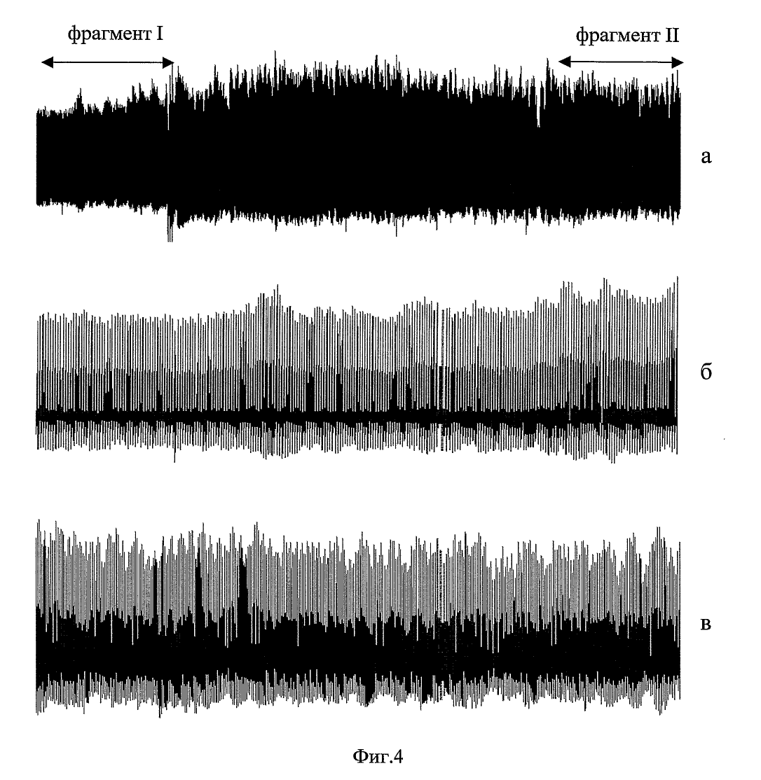

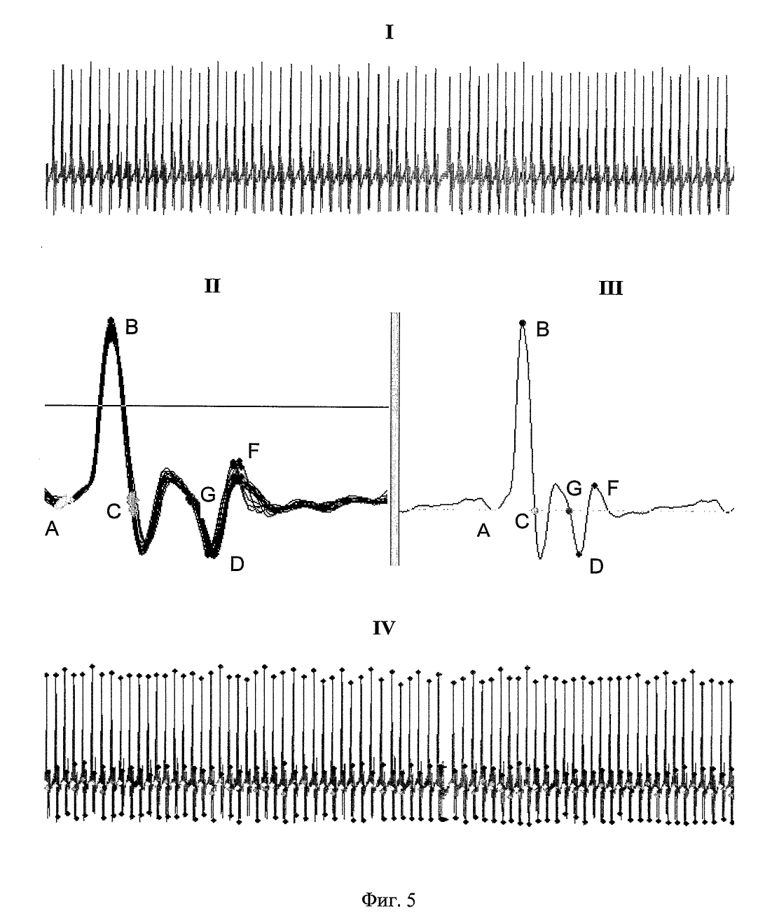

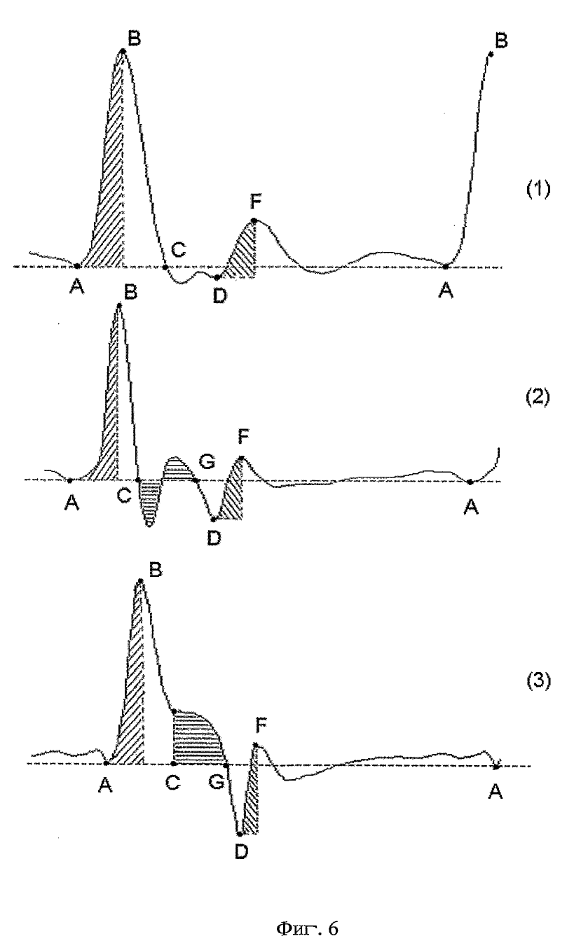

The method of pulsometric examination of the CVS will be carried out as follows (FIG. 3). The signal will be obtained in a non-invasive way from the subject under study with the aid of the piezotransducer put onto a pulsing superficial central (e.g. carotid) or peripheral (e.g. finger or temporal) artery, and the signal will be continuously recorded in the computer's operative memory. In FIG. 4a, an example is presented in the form intended for the monitor screen width graph of the 25-minute long DSPG recorded from the finger artery of the thumb of the left hand of young man (aged 23) while studying the effect of orthostatic load upon his CVS. In this form, the pulsogram will be kept in the computer hard drive memory as a file for subsequent analysis. Then the data on the patient will be entered to the file (name, age, sex, arterial pressure, past history, preliminary diagnosis, etc.) and the measurement parameters (date, time, duration of the recording, etc.). At the next stage, in compliance with the study task (in this particular case: when studying the effect of orthostatic load upon the CVS), fragments of the DSPG will be selected. In FIG. 4b, the selected 5-minute fragment of the pulsogram is presented recorded in the lying posture (fragment 1, conventional control, 5 minutes before getting up); and in FIG. 4c, a 5-minute fragment recorded in standing position (fragment 2, from the 15th to the 20th minute of the orthostatic load). These fragments lasting usually not less than 2 minutes (the standard duration being 5 minutes) can be kept in the form of separate memory files for subsequent analysis. In order to augment the precision of comparative analysis of characteristics of the separately selected DSPG fragments with the aid of the SW, temporary limits of these fragments will be set which enable one to compute the parameters strictly for them. The DSPG graph reflects the rate of AP speed changes at different stages of the cardiac cycle during the whole period of examination and represents each cardiocycle in the form of a complicated contour with characteristic inflections. This makes it possible, in compliance with the information theory and with the aid of a special computer algorithm on the DSPG graph, to single out certain points: the zero value ones (crossing/intersecting the isoline), extremal ones, and the points of inflection as the “coding” ones (reference points, supporting points) and to measure and then compute with their aid all the amplitude-temporal parameters and dimensions. Correct positioning of such points is the main condition of precision and reliability of the measurement results, and it requires a procedure of additional clarification. For this purpose, from the DSPG fragment selected as an example (FIG. 5-I), all individual pulsations will be singled out with the aid of the computer and put upon the graph of a single cardiocycle, superposing them by the coordinate of the maximum positive extremum (FIG. 5-II). Then with this set a graph of the averaged pulsation will be automatically constructed (FIG. 5-III) and, on this graph, the “coding” points will be placed and which will be checked visually by the user and, in case of an error, corrected. Normal spikes will be revealed on the DSPG graph and false spikes will be rejected from the pulsation set. To do this, the amplitude threshold will be computed (the horizontal line in the FIG. 5-II), with respect to which a search for the absolute systolic maximum will be performed (the greatest positive extremum) in the DSPG graph located above this threshold. The criterion for rejection involves a sharp deviation of the amplitude-temporal parameters of the spike under analysis from the mean values (more than for 3 root-mean-square deviations). The spike set remaining after the rejection will be considered as the set of pulsations reflecting the speed of the blood pressure changes in the subject under examination. Then the principle of proximate disposition of the points on the averaged pulsation graph will be automatically transferred onto every distinguished normal pulsation (FIG. 5-IV).

Based on the position of the “coding” points on the DSPG graph, all the temporal parameters and indices will be determined. Computation of the signal amplitude characteristics containing the information on the blood pressure value requires an additional procedure of calibrating the data for their transformation into conventional units of the AP measurement (mm Hg). To do this, the computer recording of the signal from transducer in the form of the DSPG curve will be accompanied by parallel periodic measuring of the systolic blood pressure (SBP) and diastolic blood pressure (DBP) with the aid of a sphygmomanometer. These values will be entered into the computer for computation of the mean value of the PAP (=SBP−DBP) for the selected period of examination. Correlation of this PAP value directly measured in mm Hg, with the average PAP value computed for the same period in conventional units of the computer “digitizing” by means of integrating by respective areas of the selected fragment under/over the DSPG curve, enables one to determine the calibration coefficient of the AP proportionally. Considering this coefficient, values of the pulse increment of the AP will be computed in mm Hg at different stages of the cardiac cycle, and then by them all the parameters will be computed that depend on the blood AP and characterise the cardiodynamics as well as resilient-elastic properties of the arterial bed vessel walls. This enables one to perform a long-lasting and continuous in time monitoring of the pulse oscillation pattern of the blood AP during the whole period of examination in accepted units of measurement: mm Hg. The necessary level of significance will be provided in statistical processing of the measured amplitude-temporal parameters, also it becomes possible to perform a spectral analysis of these parameters' variability, including that occurring under various effects upon the organism (loading tests, taking medicines, etc.), as well as to compare the results of the examinations fulfilled at different times.

In FIG. 6, examples of three main types of graphs for a separate cardiocycle are shown as well as versions of disposition of the “coding” points in these graphs. The types (1) and (3) of the graphs correspond to the CVS of young and elderly people, whereas the type (2) is specific for the majority of adults (aged from 25 to 55).

The DSPG graphs are the first derivatives of the graphs of time dependence of the AP change (SPG) in passing of the pulse wave which determines the single-valued disposition of the point A as a point of beginning of the anacrotic phase of the blood expulsion corresponding to the moment of the aortal valve opening. At this point, AP=DBP while the first derivative of the SPG equals zero which makes it possible to draw a horizontal line (the isoline) through this point, the line determining the area under and/or above the graph curve and reflecting increment or decrease of the blood pressure in arteries in passing of the pulse wave due to the output of the blood stroke volume expulsed from the left ventricle. The point B corresponds to the moment of reaching the maximum velocity of the AP systolic increment (the absolute positive extremum of the DSPG); the point C corresponds to the moment of reaching the maximum value of AP resulting from expulsing the blood from the left ventricle during systole (the point of crossing of the isoline by the descending portion of the pressure systolic wave curve, the first derivative of the SPG in this point being equal to zero); the point D corresponds to the moment of cessation of the blood expulsion (closing of the aortal valve, the negative extremum of the DSPG preceding the growth of the dicrotic AP) [4]; the point F corresponds to the moment of reaching the maximum velocity of the AP increment induced in the beginning of diastole by the blood pressure dicrotic wave parried from the closed aortal valve; the point G corresponds to the moment of reaching the maximum value of the secondary systolic AP increment on account of early (prior to closing of the aortal valve) and parried from the periphery primary wave of the blood pulse pressure.

Considering the “International Standards” [8] as well as methodological recommendations of a group of Russian cardiologists [9], all the main parameters will be measured and analysed by the “coding” points in the temporal area, the parameters characterising the heart rhythm and its variability: the mean duration of revealed cardiointervals (between the adjacent points “B” in FIG. 6), the mean duration of the normalised cardiointervals−TNN, as well as durations of the cardiocycle separate phases, then the variability of temporal parameters selected for measurement will be assessed (the SD, DX, CV, RMSSD, pNN50, etc. will be computed).

The “coding” points will also be used for determining (see FIG. 6) the computer “digitizing” in conventional units of the mean value of the PAP for the selected DSPG fragment: with integrating by areas covered with the ordinates between the points A and C if the area between the points C and G is lesser or equal to zero, or between the points A and G if the area between the points C and G is more than zero. As has already been noted, the comparison of this PAP value with the mean value of the PAP measured with the sphygmomanometer makes it possible to transform the conventional units of the “digitizing” into widely accepted units: mm Hg, and to compute in these units all the parameters reflecting the blood AP pulse changes in certain periods of the cardiocycle during the whole period of examination.
the value of accelerated anacrotic increment of the AP in the period of systolic output of the blood to aorta from the left ventricle−ΔAPAaccel [mm Hg] (with integrating by the area covered with ordinates between the points A and B, in FIG. 6 the area is distinguished by the hatching bent to the left);
the value of decelerated anacrotic increment of the AP in the period of systole−ΔAPAdecel [mm Hg] (with integrating by the area covered with ordinates between the points B and C or B and G, see below);
the value of dicrotic increment of the AP in the phase of its accelerated increment in the starting period of diastole−ΔAPDaccel [mm Hg] (with integrating by the area covered with ordinates between the points D and F, in FIG. 6 the area is shown by the hatching inclined to the right). This value clearly revealed in all the DSPG curves reflects the tone of the arterial bed vessel walls, the tone determining the peripheral resistance at the arteriole level which is the reason of occurrence of parried pulse waves.
the value of the secondary wavy increment of the AP on account of early and parried from the peripheral resistance wave of the pulse pressure in the period of systole−ΔAPRS [mm Hg] developing either in the catacrote period or in the phase of decelerated anacrotic blood output (as determined with integrating by the area covered with the ordinates between the points C and G).

The negative or the zero value of this area integral corresponding to the wavy catacrotic change of the AP in the DSPG graph of a single cardiocycle (in FIG. 6-2, this area is shown with the horizontal hatching) is specific for healthy adults with resilient and, at the same time, elastic walls of the aorta. In young persons, particularly in physically well-trained people with very elastic walls of the aorta, this wave will be attenuated and may be practically unnoticeable (FIG. 6-1). Positive values of the integral of the CG area (ΔAPRS more than zero) augmenting the SAP and PAP and elongating the systole decelerated anacrotic phase (in FIG. 6-3, this area is distinguished also by the horizontal hatching) indicate an excess in the normal resilience (rigidity) of the aorta walls occurring with ageing and under the effect of risk factors related to cardiovascular diseases (e.g. diabetes, smoking). This AP increment is due to the fact that the reduced elasticity of the walls hinders distention of the aorta under the effect of incoming parried from peripheral resistance wave of the blood pressure, whereas the increased velocity of wave spreading over the vascular wall [10] provides its faster return and earlier superimposition upon the primary systolic wave. Considering the above statements, in case the ΔADOC is greater than zero, in order to perform comparative assessments of the left ventricle myocardium contractile capacity in different persons under examination, the value of normalised pulse arterial pressure will be determined PAPn=PAP−ΔAPRS [mm Hg].

With these values the derivative cardiohemodynamic parameters will be computed:
the mean velocity of the AP systolic increment during the period of accelerated anacrotic blood

$$output into the aorta - VAPaccel = \frac{Δ APAaccel}{{t}_{AB}} [ mm Hg \text{/} s ] ,$$

where t_{AB}−duration of the AB period,
the maximum velocity of this increment VmaxAPA [mm Hg/s] as determined by the ordinate of the point B.

The blood pressure pulse wave parried from the peripheral resistance does not affect (does not superimpose itself upon) these values and, therefore, they, together with the normalised PAPn value, reflect just the left ventricle myocardium contractile capacity and the left valve condition, i.e. characterise efficacy of the pumping (forcing) function of the heart;

$$- cardiohemodynamic index - CHDI = \frac{Δ APAaccel}{Δ APAdecel} ,$$

which also characterises efficacy of the left ventricle myocardium contractile (pumping) function and may serve as an indicator of developing stenosis of the aortal valve and of growing rigidity of the aorta walls. The parameters will be determined which characterise resilient-elastic properties of the vessel walls in the arterial bed:

$$- the rigidity index of the aorta walls - RIA = \frac{Δ APRS}{PAPn} \cdot 100 \% ,$$

if ΔAPRS is greater than zero;

$$- tone index of the artery walls - TIA = \frac{Δ APDaccel}{Δ APAaccel} ,$$

where ΔAPDaccel−the accelerated dicrotic increment of the blood AP in the starting period of diastole.

As an example, in Table 1, the results are shown that have been obtained with the aid of the applied method and characterise the effect of orthostatic load upon cardiac rhythm, cardiohemodynamics and resilient-elastic properties of the arterial bed vessels in practically healthy young men (the subject-I, aged 23) and elderly men (the subject-II, aged 69). The results obtained show that the orthostatic load affects the CVS functional condition in young as well as in elderly men, the character of this influence changing with age. The load leads to increment of the heart rate in both subjects under study, but in the young man, this increment is more obvious and is accompanied by augmentation in the rhythm variability (the amplitude of the duration mode of the NN intervals in the histogram is considerably lower). In both subjects, while preserving the mean velocity of the AP anacrotic increment in the phase of accelerated expulsion of the blood from the left ventricle, the maximum velocity of the AP increment is significantly increased. At the same time, a difference in direction of the cardiohemodynamic index changes (CHDI) becomes obvious in the subjects under study: in the first one (the CHDI dropped to 1.08 from 1.51), with preserved value of the normalised PAP, the orthostatic load resulted in redistribution of the relative contribution of accelerated and decelerated AP increments on account of an increase in the share of the ΔAPAaccel (from 28 to 24 mm Hg); in the second subject under study (the CHDI increased to 0.69 from 0.47), under the conditions of the load, the necessary level of the PAPn was preserved through an augmentation of the relative contribution of ΔAPAaccel (from 15 to 19 mm Hg). On the basis of these single-patient data given as an example, one may suppose that the noticed age-bound changes of the cardiohemodynamics parameters are functionally conjugated with the changes of the resilient-elastic properties of the arterial bed vessel walls in the subjects under study. In young man, the walls of both aorta and arteries are elastic (the RIA value is less than zero), and the adequate circulation under the load will be provided by an increase in the tone of the artery walls (the TIA value increases from 0,308 to 0,743). In the elderly person with rigid walls of the vessels, the adequate circulation under the orthostatic load will be provided by a drop in the vascular tone (the TIA value drops to 0.497 from 0.632). This example illustrates the advantages of using the applied method as compared, for instance, with widely accepted in the cardiological practice ECG technique whose capacities are limited by obtaining information on just temporal characteristics of the cardiac rhythm.

Using the Fourier processing algorithm, spectral analysis of the DSPG curve will be performed both by the heart rhythm variability (the changeability of the NN interval durations−TNN) and by the variability of parameters characterising the cardiohemodynamics and arterial vessel tonus: PAPn, VmaxAPA, ΔAPDaccel, etc., depending on the study goal. Table 2 shows results of the spectral analysis of the heart rhythm variability (by the TNN value) and of the normalised pulse arterial pressure (PAPn) in the young (I) and elderly (II) subjects. It shows that, under the conditions of orthostatic load of the organism, the contribution of the VNS sympathetic link to regulation of the heart rhythm increases relative to the parasympathetic effect, and in the young man this redistribution is considerably more obvious than in the elderly man (the index of the sympatho-vagal balance: SVI, increases from 1.5 to 8.8 in the first subject and only from 2.6 to 3.9 in the second subject). The revealed changes of the sympatho-vagal balance of the PAPn vegetative regulation in these subjects were less significant in their extent and had opposite sign. The orthostatic load did not considerably affect the summed up spectral power (TP) of the young man's PAPn variability, whereas in the elderly man it increased the TP more than 2-fold (from 10.3 to 24.0 [mm Hg]²). One can see that, in both subjects under conditions of the orthostatic load, a considerable redistribution of the relative participation of regulatory systems occurs in the course of maintenance of the hemodynamics necessary level. The relatively slow humoral-metabolic regulation revealed in the laying posture within the main range of the ULF (70% and 65% of the TP values in I and II subjects, respectively) will be replaced by a faster neurogenic regulation (within the LF range, for instance, the spectral power of the PAPn value oscillations increases in the I subject from 11% to 41%).

Thus, the applied method extends the range of studying the character of autonomic and humoral-metabolic regulation of the human CVS, thus opening up new possibilities of studying the physiological mechanisms of the circulation control based on the control not of the heart rhythm alone but of the hemodynamic parameters related to the blood AP pulse changes, too. Juxtaposition of results of the spectral analysis of various parameters' variability enables one to obtain qualitatively new information on the role and relative contribution of the sympathetic and parasympathetic portions of the autonomic nervous system, as well as other regulatory systems for homeostasis to the regulation both of the cardiac rhythm and of the functional characteristics of the myocardium and the smooth-muscle structures of the arterial bed vessel walls which jointly determine the dynamics of the AP pulse change for providing a physiologically adequate circulation.

The results of statistical as well as spectral analysis of the measured parameters' variability (the parameters being selected in dependence on the study objective) help to assess the functional condition and the character of the CVS vegetative regulation in the subjects under study by means of comparison of the parameters' measured values with the average statistical numerical values of the same parameters as they were found for the CVS of groups of subjects classified by age, sex, health condition (past history) and environmental conditions, the groups having been selected as the control. Based on use of special techniques of the statistical analysis (discriminant, dispersion, or factorial), these results can be used for resolving problems of differential diagnosis of the patients' CVS condition.

### INDUSTRIAL APPLICABILITY

The use of a piezoceramic transducer in combination with the use of the computer recording and analysis of the DSPG made it possible to develop a simple in use automated method of precise quantitative express-analysis of a wide range of some known and some new parameters jointly characterising the functional condition of the CVS as well as specifics of its regulation by the ANS. On the basis of the developed method, an arterial piezopulsometer can be manufactured which, in the form of an autonomous compact and inexpensive attachment to computer or in the form of a component of universal multifunctional system of cardioscreening, could satisfy the demand of domestic clinics, diagnostic and sporting-rehabilitative centres, specialised sanitary divisions and similar medical institutions for such devices. The simplicity of servicing the autonomous version of the pulsometric attachment for a PC will make it possible to use the device for regular individual examination of patients as well as for large-scale observation of the CVS condition in various groups of population (e.g. students, military servicemen, workers at high risk enterprises, the work force in remote places, etc.). The proposed method makes it possible to perform an operative check-up of the human CVS condition under stress effects, under conditions of unfavourable ecological situation, as well as monitoring of the CVS condition in professionals associated with continuous and stressed work: air traffic controllers, pilots, astronauts, etc.

### SOURCES OF INFORMATION

1. RF U.S. Pat. No. 2,200,461“Method of diagnosis by the cardiorhythm and the device for performing it” (Aldonin G. M. et al.), published on Mar. 20, 2003
2. Langewouters G. J., Settels J. J., Roelandt R., Wesseling K. H. Why use Finapres or Portapres rather than intra-arterial or intermittent non-invasive techniques of blood pressure measurement//J Med Eng Technol. 1998. Vol. 22. P. 37-43.
3. RF U.S. Pat. No. 2,013,990“Method of assessment of the cardiovascular system functional condition” (Sheikh-Zade Yu. R. et al.), published on Jun. 15, 1994
4. N. R. Paleev and I. M. Kaevitser. The atlas of charts of hemodynamic studies at internal disease clinic.—M.: “Meditsina”, 1975. pp. 238
5. V. S. Logvinov. Method of diagnosis by the parameters of oscillatory and wave-form processes in the cardiovascular system//In.: The pulse diagnosis in Tibetan medicine.—Novosibirsk: “Nauka”, 1988. pp 90-98.
6. V. V. Boronoev, V. D. Dashinimaev, E. A. Trubachev. Pulse transducers for practical diagnosis in Tibetan medicine//In.: Pulse diagnosis in Tibetan medicine.—Novosibirsk: “Nauka”, 1988. pp 64-77.
7. A. V. Samoilenko, V. A. Orlov. Use of computation methods and simulation in studying the cardiovascular system//In.: Methods of studying the circulation.—Leningrad: “Nauka”, 1976. pp 241-270.
8. Heart rate variability. Standards of measurement, physiological interpretation, and clinical use//European Heart Journal. 1996. Vol. 17, P. 354-381
9. R. M. Baevsky, G. G. Ivanov, L. V. Chireikin et al. Analysis of the heart rhythm variability while using different electrocardiographic systems—Moscow. 2002. pp 50.
10. N. N. Savitsky Biophysical elements of circulation and clinical methods of studying the hemodynamics—Leningrad: “Meditsina”, 1974. pp 311

TABLE 1
Influence of orthostatic loading on parameters of the functional condition of cardiovascular
system of young and aged men

| | Patient -I | | Patient -II | |
|---|---|---|---|---|
| | (23 old) | | (69 old) | |
| Parameters | Lying | Stand | Lying | Stand |
| CardioRhythm: | | | | |
| Frequency of cardio contraction (FCC), pound/min | 56 | 80 | 57 | 61 |
| Mean duration of normalised cardiointervals (TNN), ms | 1070 | 750 | 1060 | 980 |
| Standard deviation NN of intervals (SDNN), ms | 51 | 59 | 26 | 21 |
| Mode NN of intervals (MoNN), ms | 1080 | 765 | 1045 | 970 |
| Amplitude of mode of NN-intervals (AMoNN), % | 44.1 | 28.0 | 67.5 | 51.5 |
| Fraction of different adjacent intervals (pNN50), % | 30.8 | 8.9 | 0 | 0 |
| Cardiohemodynamics: | | | | |
| Normalized PAP (PAPn), mm Hg/s | 47 | 47 | 45 | 46 |
| Standard deviation PAPn (SD PAPn), mm Hg/s | 5.5 | 4.6 | 5.2 | 7.5 |
| Accelerated anacrotic increment AD (ΔΔDAaccel.), mm Hg/s | 28 | 24 | 15 | 19 |
| Mean velocity of increment ADA (VADAaccel.), mm Hg/s | 335 | 352 | 210 | 214 |
| Maximum velocity of this increment PAP (VmaxADA), mm Hg/s | 703 | 801 | 437 | 519 |
| Cardiohemodynamic index CHDI | 1.51 | 1.08 | 0.47 | 0.69 |
| Resilient-elastic properties of the arterial bed vessels | | | | |
| Rigidity index of aorta walls (RIAo), % | — | — | 38.5 | 23.2 |
| Arterial wall tone index (TIA) | 0.308 | 0.743 | 0.632 | 0.497 |

TABLE 2
Influence of orthostatic loading on the spectrum analysis parameters characterizing the
features of vegetative regulation of cardiovascular system of young and aged men

| | Patient-I (23 old) | | Patient-II (69 old) | |
|---|---|---|---|---|
| Parameters of spectrum analysis of variability | Lying | Stand | Lying | Stand |
| a) of heart rhythm: | | | | |
| Cumulative spectrum power (TP), ms² | 4000 | 6681 | 1297 | 885 |
| Spectrum power of high frequencies (HF), ms² | 1255 | 407 | 100 | 77 |
| Spectrum power of high frequencies (HF), % | 31 | 6 | 8 | 9 |
| Spectrum power of low frequencies (LF), ms² | 1845 | 3576 | 258 | 296 |
| Spectrum power of low frequencies (LF), % | 46 | 54 | 20 | 33 |
| Spectrum power super-low frequencies (VLF), ms² | 391 | 1738 | 456 | 203 |
| Spectrum power super-low frequencies (VLF), % | 10 | 26 | 35 | 23 |
| Spectrum power ultralow frequencies (ULF), ms² | 510 | 960 | 483 | 309 |
| Spectrum power ultralow frequencies (ULF), % | 13 | 14 | 37 | 35 |
| Sympathovagal parameter (index) (SVI) | 1.5 | 8.8 | 2.6 | 3.9 |
| 6) of PAPn magnitude: | | | | |
| Cumulative spectrum power (TP), [mm Hg]² | 29.6 | 31.9 | 10.3 | 24.0 |
| Spectrum power of high frequencies (HF), [mm Hg]² | 2.9 | 8.5 | 1.1 | 8.0 |
| Spectrum power of high frequencies (HF), % | 9.9 | 27 | 11 | 33 |
| Spectrum power of low frequencies (LF), [mm Hg]² | 3.2 | 13.1 | 1.8 | 9.9 |
| Spectrum power of low frequencies (LF), % | 11 | 41 | 17 | 41 |
| Spectrum power super-low frequencies (VLF), [mm Hg]² | 2.7 | 7.2 | 0.7 | 1.9 |
| Spectrum power super-low frequencies (VLF), % | 9.2 | 23 | 7 | 8 |
| Spectrum power ultralow frequencies (ULF), [mm Hg]² | 20.8 | 3.1 | 6.7 | 4.2 |
| Spectrum power ultralow frequencies (ULF), % | 70 | 10 | 65 | 18 |
| Sympathovagal parameter (index) (SVI) | 1.1 | 1.5 | 1.6 | 1.2 |

1. A method for pulsometric assessment of the functional state and the character of autonomic regulation of the human cardiovascular system wherein, from the subject under study in relative resting and under conditions of performing a load test, a pulsogram is recorded in a non-invasive way by the method of differential sphygmography with the aid of a respective transducer, the pulsogram being studied using the method of the “coding points”, characterized in that the pulsogram analogue signal picked off from the transducer is transformed into a digital signal continuously recorded and analysed with the aid of a computer, with parallel periodic measurement of the blood arterial pressure (AP) using a sphygmomanometer; then a pulsogram fragment at least 2-minute long is selected and used for constructing a graph of the averaged cardiocycle and determining the “coding points”; the principle of the “coding points” disposition is transferred to each recognised normal pulsation of the selected fragment; and then, by the fragment, the temporal parameters characterising the heart rhythm and its variability are measured; by the same pulsogram fragment, the average value of the pulse arterial pressure (PAP) in conventional digital units is determined by means of integrating a respective part of the differential sphygmogram curve; and then, comparing this value with the average PAP value measured at the same period with the sphygmomanometer, the calibration coefficient of the AP proportionality is determined; and, taking the calibration coefficient into account, the conventional digital units are re-computed into conventional mm Hg, and on their basis the values of the blood arterial pressure increment are computed for different stages of the cardiac cycle, these values then being used for determining the amplitude-temporal cardiohemodynamic parameters characterising the left ventricle myocardium contractile capacity, namely:
normalised pulse arterial pressure−PAPn;
$$- cardiohemodynamic index = CHDI = \frac{Δ APAaccel}{Δ APAdecel} ,$$
where ΔAPAaccel and ΔAPAdecel are, respectively, accelerated and decelerated anacrotic increments of the arterial pressure;
the average−VAPAaccel and maximum−VmaxAPA velocities of the anacrotic increment of the AP in the phase of accelerated expulsion of the blood from the left ventricle;
and the resilient-elastic properties of the arterial bed vessel walls, such as:
$$- rigidity index of the aorta walls RIA = \frac{Δ APRS}{PAPn} \cdot 100 \% ,$$
where ΔAPRS is the arterial pressure increment due to the pulse pressure wave parried from the periphery during the systole period, and
$$- the arterial walls tone index - TIA = \frac{Δ APDaccel}{Δ APAaccel} ,$$
where ΔAPDaccel is the accelerated dicrotic increment of the blood arterial pressure during the starting period of diastole;
after which statistical processing of all the parameters is performed and spectral analysis of the heart rhythm variability and the selected amplitude-temporal cardiohemodynamic parameters are fulfilled, the results of which make it possible to assess the functional condition and specifics of the autonomic regulation of the subject's cardiovascular system by means of comparing the obtained parameters with the average statistical reference values of the same parameters as established for reference groups of subjects classified by age, sex, health condition and other signs.
